(19) United States
(12) Patent Application Publication
(10) Pub. No.: US 2009/0192566 A1
(43) Pub. Date: Jul. 30, 2009

(54) Device for and Method of Generating a Baroreflex Response

(75) Inventor: Kok-Hwee Ng, Stow, MA (US)

Correspondence Address: MARSHALL, GERSTEIN & BORUN LLP, 233 SOUTH WACKER DRIVE, 6300 SEARS TOWER, CHICAGO, IL, 60606-6357, US

(73) Assignee: STRYKER DEVELOPMENT LLC, Cambridge, MA (US)

(21) Appl. No.: 12/020,267

(22) Filed: Jan. 25, 2008

(51) Int. Cl. A61N 1/36

(52) U.S. Cl. 607/44

(57) ABSTRACT

This patent relates to a device for and method of stimulating the carotid sinus baroreceptors to achieve a restoration or resetting of baroreflex response and natural blood pressure modulation. The stimulation may consist of low amplitude, wide bandwidth signals that produce a stochastic resonance effect. The ultimate affect is blood pressure modulation within a reduced range including establishment of thresholds at which baroreceptors trigger normal central nervous system functions.

### TECHNICAL FIELD

This patent relates to baroreflex regulation through electronic stimulation, and in particular, this patent relates to a device and a method to improve baroreflex response in a subject.

### BACKGROUND

The baroreflex control of arterial blood pressure operates primarily through the carotid sinus baroreceptors that respond to arterial blood pressure changes with afferent action potential to the central nervous system (CNS). The CNS in turn makes adjustments to sympathetic and parasympathetic efferent activities appropriately to accommodate the demand of the original arterial pressure change. In hypertension and a number of cardiovascular diseases (such as sleep apnea and cardiac failure), the baroreflex sensitivity of the patient is believed to be impaired. These patients lack the ability to respond to arterial pressure changes as quickly as normal subjects would, and are said to have their barorelex reset to higher blood pressure levels.

The cause of hypertension is multi-factorial. One factor that is commonly attributed to is the stiffening of the carotid artery, which comes with ageing and atheroscleroris. In normal carotid arteries, the pulsing of blood pressure causes the diameter of the artery to distend and contract according to the pulse pressure. The change in diameter stretches the baroreceptors on the arterial wall and generates a potential proportional to the amount of distension. Some of these generator potentials result in action potentials, but some do not due to varying degree of baroreceptor thresholds. The action potentials are the input to the CNS to regulate the appropriate level of sympathetic and parasympathetic output based on the body's physiological need at the time. For example, when a person goes from a sitting to a standing posture, there is a transient drop in blood pressure when blood is drawn to the lower extremities. The lowering of blood pressure reduces the distension of the carotid artery, which in turn reduces baroreceptor activity. The CNS responds by reducing the parasysmpathetic output and increasing the sympathetic output, thereby increasing the heart rate. As a result, blood flow is increased and blood pressure returns to the level of the seated posture.

The stiffening of the carotid artery reduces the amount of distension possible at each blood pressure level, so that a healthy level of baroreceptor activity can only be achieved at a higher blood pressure. This is referred in the literature as the resetting of blood pressure in chronic hypertension.

Traditional treatment of hypertension has focused on pharmaceutical approaches to regulate blood volume. Typically, a cocktail of drugs are used to achieve the desired blood pressure target, with associated side effects.

A device approach to provide direct baroreflex stimulation to treat hypertension has been proposed. The device consists of an implantable electrode with a subcutaneously placed controller. It is intended to target treatment resistant hypertension patients by constantly stimulating the carotid sinus baroreceptors at a fixed rate sufficient to generate a response. Although the device may be effective in achieving specific targets of blood pressure reduction, it does not address the dynamic demands of the cardiovascular system in all physical conditions. The possibility of adaptation of the baroreflex system to constant frequency stimulation is also a concern. An additional side affect is undesired stimulation of nearby anatomical structures including vagal nerve stimulation, as well as somatosensory nerve stimulation resulting in pain and irritation. Electrode migration and over time erosion can damage arterial and nerve tissues.

A further limitation of direct nerve stimulation devices, such as of the type described above, relates to baroreceptor afferent nerve activities. Research shows baroreceptor afferent nerve activities occur in synchronous with the arterial pulse pressure cycle, such that more carotid sinus nerve action potentials occur during the systolic phase compared to the diastolic phase of the cycle. This is consistent with the different extent of arterial wall stretch between the systolic and diastolic phases. In addition, it has also been suggested that the baroreceptor afferent nerve activities are proportional to the first derivative of the instantaneous pulse pressure. In other words, the faster the pressure change the higher the nerve activities. For direct nerve stimulation device to maintain a range of normal blood pressures, it would have to generate stimulation pulses that mimic the natural baroreceptor nerve activities that occur in each pulse pressure cycle. The complexity to achieve this would be exponentially large given the dynamic demands of the body under different physiological conditions (e.g., awake vs sleeping vs exercise).

Neither pharmaceutical nor device treatment of hypertension is effective to restore the baroreceptor sensitivity of a hypertension patient to respond to dynamic cardiovascular demands so that the resetting of blood pressure is naturally reversed. That is, none of the known treatments for hypertension provide for restoring baroreflex activity to affect normal blood pressure modulation.

### BRIEF DESCRIPTION OF THE DRAWINGS

FIG. 1 illustrates a human torso and related vasculature and a device in accordance with an embodiment of the invention

FIG. 2 is a block diagram illustration of a device controller in accordance with an embodiment of the invention

FIG. 3 illustrates a human torso and related vasculature and a device in accordance with an embodiment of the invention

FIG. 4 is a graphic illustration of a baroreflex response threshold and a baroreflex generator potential.

FIG. 5 is a graphic illustration of a baroreflex response threshold and a baroreflex generator potential responsive to stimulus in accordance with a preferred embodiment of the invention.

Skilled artisans will appreciate that elements in the figures are illustrated for simplicity and clarity. It will further be appreciated that certain actions and/or steps may be described or depicted in a particular order of occurrence while those skilled in the art will understand that such specificity with respect to sequence is not actually required. It will also be understood that the terms and expressions used herein have the ordinary meaning as is accorded to such terms and expressions with respect to their corresponding respective areas of inquiry and study except where specific meanings have otherwise been set forth herein.

### DETAILED DESCRIPTION

While the invention described by this patent is susceptible to various modifications and alternative forms, certain embodiments are shown by way of example in the drawings and these embodiments will be described in detail herein. It should be understood, however, that this disclosure is not intended to limit the invention to the particular forms described, but to the contrary, the invention is intended to cover all modifications, alternatives, and equivalents falling within the spirit and scope of the invention defined by the appended claims.

This patent relates to a device for and method of stimulating the carotid sinus baroreceptors. A beneficial outcome of the use of a device or practice of a method and in particular a stimulation of the carotid sinus baroreceptors in accordance with one or more of the herein described embodiments of the invention is restoration or resetting of baroreflex response and natural blood pressure modulation. A preferred stimulation may consist of wide bandwidth signals that produce a stochastic resonance effect. The ultimate affect is blood pressure modulation within a reduced range including establishment of thresholds at which baroreceptors trigger normal CNS functions.

In accordance with an embodiment of the invention, a fully external, a partially implantable or a fully implantable device is capable of delivering one or more forms of energy to the carotid sinus baroreceptors. The energy may be delivered at an intensity and frequency to effect restoration of baroreflex response.

In an embodiment of the invention providing a fully externally deployed device, electrodes may be positioned within a collar or a collar portion of an article of clothing or garment to be worn by the patient. Incorporation of the electrodes within a collar or collar portion of a garment ensures therapeutically effective location of the electrodes relative to the carotid sinus baroreceptors. The electrodes may be suitably coupled, wired or wirelessly, to a controller that may be worn within the garment or elsewhere on the patient.

In an embodiment of the invention providing a partially implanted device, the electrode may be subcutaneously implanted adjacent the baroreflex receptors. To remove the requirement to provide a wired connection to the electrodes, the electrodes may be configured to be energized and respond to a remotely generated stimulus to provide the desired stimulation. A suitable controller may be worn on or carried by the patient. An alternative approach may provide for an implanted electrode and controller, wherein the controller may be activated by an externally provided signal to affect charging, programming or other desired functionality.

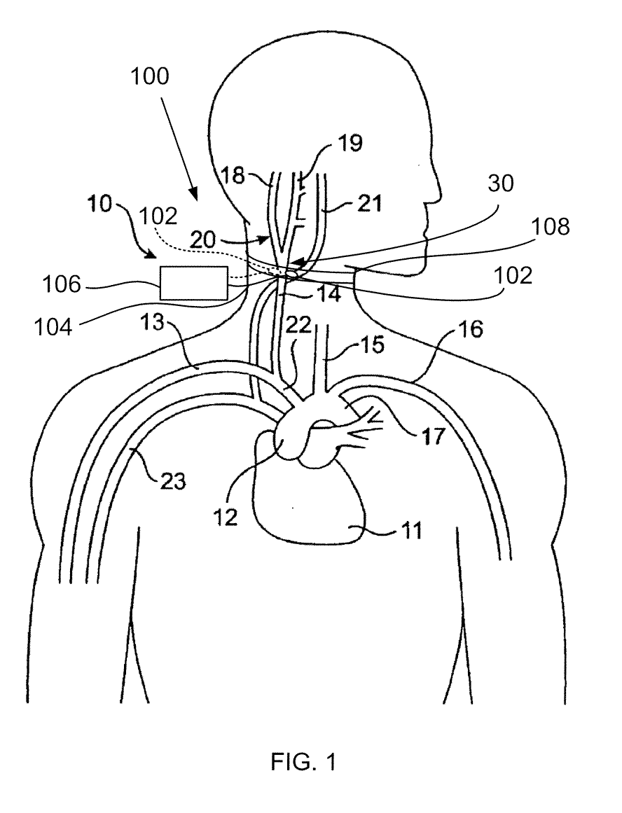

FIG. 1 illustrates the basic vascular anatomy associated with the cardiovascular system within a schematic illustration of the upper torso of the human body 10. The left ventricle of the heart 11 pumps oxygenated blood up into the aortic arch 12. The right subclavian artery 13, the right common carotid artery 14, the left common carotid artery 15 and the left subclavian artery 16 branch off the aortic arch 12 proximal of the descending thoracic aorta 17. Although relatively short, a distinct vascular segment referred to as the brachiocephalic artery 22 connects the right subclavian artery 13 and the right common carotid artery 14 to the aortic arch 12. The right carotid artery 14 bifurcates into the right external carotid artery 18 and the right internal carotid artery 19 at the right carotid sinus 20. The left carotid artery 15 similarly bifurcates into the left external carotid artery and the left internal carotid artery at the left carotid sinus (not depicted).

From the aortic arch 12, oxygenated blood flows into the carotid arteries 18/19 and the subclavian arteries 13/16. From the carotid arteries 18/19, oxygenated blood circulates through the head and cerebral vasculature and oxygen depleted blood returns to the heart 11 by way of the jugular veins (only the right jugular vein 21 being depicted). From the subclavian arteries 13/16, oxygenated blood circulates through the upper peripheral vasculature and oxygen depleted blood returns to the heart by way of the subclavian veins (only the right subclavian vein 23 being depicted). The heart 11 pumps the oxygen depleted blood through the pulmonary system where it is reoxygenated. The re-oxygenated blood returns to the heart 11, which pumps the re-oxygenated blood into the aortic arch to repeat the cycle.

Within the arterial walls of the aortic arch 12, common carotid arteries 14/15 (near the right carotid sinus 20 and left carotid sinus), subclavian arteries 13/16 and brachiocephalic artery 22 there are baroreceptors. For example, baroreceptors 30 reside within the vascular walls of the carotid sinus 20. Baroreceptors are a type of stretch receptor used by the body to sense blood pressure. An increase in blood pressure causes the arterial wall to stretch, and a decrease in blood pressure causes the arterial wall to return to its original size. Such a cycle is repeated with each beat of the heart. Because baroreceptors 30 are located within the arterial wall, they are able to sense deformation of the arterial wall and adjacent tissue, which is indicative of a change in blood pressure. The baroreceptors 30 located in the right carotid sinus 20, the left carotid sinus and the aortic arch 12 are believed to play the most significant role in sensing blood pressure that affects the baroreflex system.

The baroreceptors 30 are diffusely disposed in the vascular wall of the major arteries. Each baroreceptor 30 transmits baroreceptor signals to the brain via nerve tissue of the central nervous system. The signals are used to activate a number of body system collectively referred to as the baroreflex system. Specifically, the baroreflex system initiates a sequence to increase heart rate and increase contraction force in order to increase cardiac output; alter kidney function and vessel constriction all to increase blood pressure and to reverse this sequence to reduce blood pressure.

An external, implantable or combination device in accordance with the preferred embodiments of the invention is capable of delivering one or more forms of energy to the carotid sinus baroreceptors 30 such that the frequency of baroreflex response is restored. As depicted in FIG. 1, a device 100 for stimulating the carotid sinus baroreceptors 30 includes pairs of electrodes 102 positioned externally and adjacent the left and right carotid sinus baroreceptor 30. A single block illustrates the right electrode pair 102 in solid line and a single block illustrates the left electrode pair 102 depicted in phantom for the sake of clarity. Single electrodes may replace the electrode pairs, and more than two electrode pairs or single electrodes may be used. Potentially fewer than two electrode pairs, i.e., one electrode pair or a single electrode, may be used. To facilitate placement of the electrodes closely adjacent the carotid sinus baroreceptor 30, the electrodes 102 may be incorporated into a collar 108 that may be worn about the patient's neck, comfortably. In this regard, the collar 108 may be custom fitted for the patient or made adjustable in size and/or from elastic material. The electrodes 102 may be coupled by wires 104 to a controller 106.

Conveniently, the collar 108 may be made as part of a garment, such as a shirt or jacket (not depicted). In such an embodiment, the controller 106 and wires 104 may likewise be made as part of the garment. For example, the electrodes 102 may be sewn, woven, bonded or otherwise secured within a collar portion of the garment. Similarly, the wires 104 may be woven or sewn into the fabric of the garment. The garment may be made with a pocket, loops or straps to receive and secure the controller 106, although one will appreciate with the ever decreasing size of electronics and power sources, the controller 106 too may be sewn or woven into the fabric of the garment. In such an embodiment, the patient needs only to dress in the garment comfortably fastening the collar 108 about their neck and actuate the controller 54 to receive therapy. Alternatively, radio frequency transmission may couple the controller 106 and the electrodes 102 to power and control them thus eliminating the need for the wires 104.

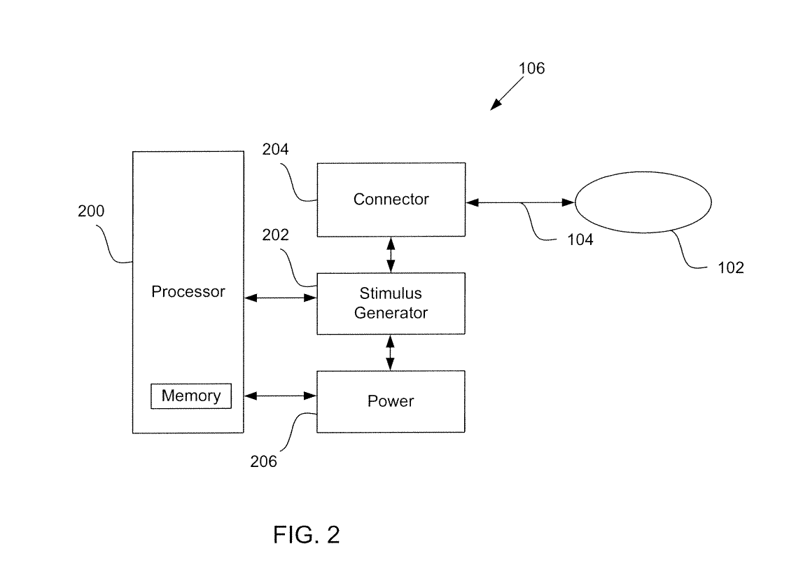

The block diagram illustrated in FIG. 2 shows the operative elements of the device 100. While shown as separate elements, it will be appreciated that the functional elements may be made as a single, self-contained device. In fact, much of the inventive functionality and many of the inventive principles described herein are best implemented with or in software programs or instructions and integrated circuits (ICs) such as application specific ICs. It is expected that one of ordinary skill, notwithstanding possibly significant effort and many design choices motivated by, for example, available time, current technology, and economic considerations, when guided by the concepts and principles disclosed herein will be readily capable of generating such software instructions and programs and ICs with minimal experimentation. Therefore, in the interest of brevity and minimization of any risk of obscuring the principles and concepts in accordance to the present invention, further discussion of such software and ICs, if any, will be limited to the essentials with respect to the principles and concepts of the preferred embodiments.

As depicted in FIG. 2, the controller 106 may include a processor 200, such as a general purpose processor, an application specific processor or any suitable processing device capable of operating on a set of instructions and/or data stored within a memory thereof (the memory, instructions and data may be static or dynamic) to implement the herein describe functionality. The processor 200 couples to a stimulus signal generator 202 and via an optional connector block 204 and wires 104 to the electrodes 102. An electrical power source 206 provides electrical energy. The electrical power source may be a battery pack such as a rechargeable battery of suitable chemistry like nickel cadmium (NiCad); nickel-metal hydride (NiMH); Lithium Ion (LiIon) or replaceable non-rechargeable batteries. Alternatively, the power source 206 may be a chemical fuel cell; a power source adaptor for connecting to a power grid or the like.

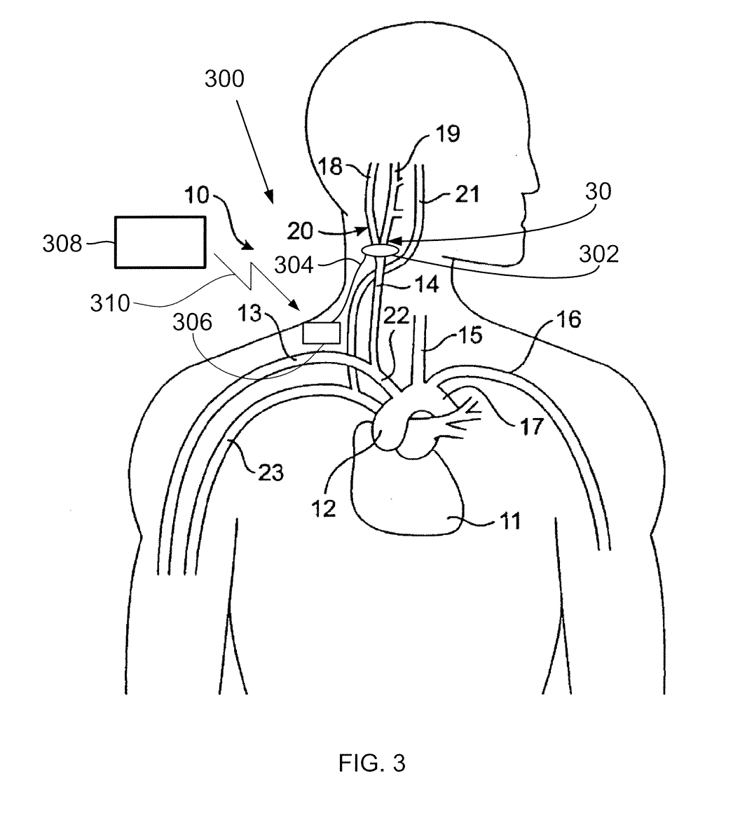

FIG. 3 illustrates a device 300 for stimulating the carotid sinus baroreceptors 30. The device 300 includes one or more subcutaneously implanted, thin film electrodes, one of which is illustrated as electrode 302. Typically two electrodes 302 are used, one each positioned adjacent the left and right carotid sinus baroreceptors 30, respectively. The electrode 302 couples via subcutaneously implanted conductors, e.g., wires, 304 to a subcutaneously implanted controller 306. The device 300 essentially operates the same as the device 100, with the controller likewise incorporated essentially the same components of the controller 106 as depicted in FIG. 2.

An external charging/programming device 308 may be provided. The device 308 provides a signal 310 of suitable frequency and power to excite a receiver circuit (not depicted) within the controller 306. Such receiver circuits are well known, particularly in the field of remotely powered and interrogated radio frequency identification (RFID) tags, and a discussion of how power is transferred to the controller 306 is well within the skilled artisan. The device 308 therefore is capable of maintaining the charge of the controller 306 battery pack. Additionally, the device 308 is capable of communicating programming instructions to the controller 306 to affect changes in its operating program and hence overall operation of the device 300. Furthermore, the controller 306 may also be capable of communicating data, such as operating or diagnostic data, to the device 308 to assist maintenance and repair functions.

In an alternative embodiment, the functions of the controller 306 may be integrated with the electrode 302. The device 308 may then both power and control the electrode 302/controller 306 combination. The electrode 302/controller 306 combinations may be configured to operate using stored energy or in the presence of the external device 308 or both.

The device 100 or 300 or any device configured to operate in accordance with the herein described embodiments, provide a stimulus to the carotid sinus baroreceptors 30 intended to enhance only nerve activities that are already initiated by the body in association with specific tasks. In this way, the stimulus is unlikely to cause undesirable nerve activities in nearby tissue structures in the absence of task initiation. It is possible the stimulus may enhance other nerve activities (either excitory or inhibitory) along with baroreceptor initiated activities. It is believed the sum effect of all enhancements is likely to be beneficial as compared to direct nerve stimulation techniques.

The stimulus waveform may be a random noise waveform of amplitudes and frequencies below the sensory perception of the patient. By itself, the stimulus has no effect on the physiology. In this regard, it is sub-threshold and hence does not have sufficient energy to directly stimulate baroreflex response. However, in the presence of a sub-threshold generator potential resulting from arterial blood pressure changes at the baroreceptors 30, the addition of low amplitude noise is sufficient to cause the baroreceptor generator potential to exceed the threshold and trigger an action potential. Therefore, the action potentials that have been suppressed at hypertensive conditions can now be called into action to regulate the blood pressure back to a normal level. It is believed that continuous use of a device, such as device 100 or 300, can result in long term restoration of the arterial blood pressure to a normal range.

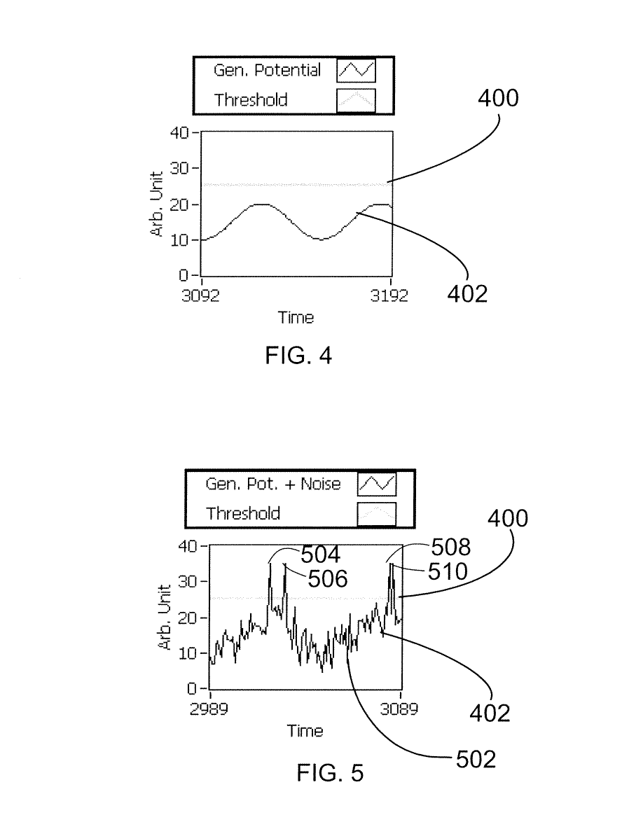

The graph of FIG. 4 schematically illustrates a threshold 400 and a baroreceptor generator signal 402, depicted below the threshold 400 and in the absence of action potentials. Without further excitation, e.g., an action potential, the generator signal 402 will not cause baroreflex response affecting blood pressure.

The graph of FIG. 5 schematically illustrates the same threshold 400; however, the baroreceptor generator signal 402 is enhanced by a random white noise signal 502. Peaks of the sine wave signal 402 insufficient to exceed the threshold 400 now do exceed the threshold 400, resulting in action potentials 504, 506, 508 and 510. The result is a baroreflex response to the generator potential affecting blood pressure. Furthermore, an advantage of the proposed indirect low level noise stimulation is that it is applied independent of the phase of the pulse pressure, and yet results in afferent nerve activities that are in synchronous with the pulse pressure and hence the physiological demands at the instant (e.g., the sine wave contour of FIG. 4 is maintained despite the addition of noise as depicted in FIG. 5).

From the description of embodiments of devices for providing baroreflex stimulation and the discussion associated with FIGS. 4 and 5, a methodology of controlling blood pressure may be envisioned. The method may include initiating a natural baroreflex response by providing a low level stimulus to one or both of the carotid sinus baroreceptors 30. The effect of the low level stimulation is to cause peaks of normally occurring baroreflex generator potential to exceed a threshold resulting in a baroreflex response.

It will be appreciated that numerous variations to the above-oaches mentioned approaches are possible. Variations to the above approaches may, for example, include increasing or decreasing the numbers of electrodes used, the manner of positioning the electrodes adjacent the baroreceptors, the stimulated baroreceptors; the form of and energy of the stimulus, and the like. For example, the noise stimulus level may be tuned to each patient's target blood pressure range, since stochastic resonance effect follows an inverted signal to noise ratio curve. A basic stimulus may be modified as part of a configuration step during a device fitting/patient screening or configuration step.

All references, including publications, patent applications, and patients, cited herein are hereby incorporated by reference to the same extend as if each reference were individually and specifically indicated to the incorporated by reference and were set forth in its entirety herein.

Preferred embodiments of this invention are described herein, including the best mode known to the inventor for carrying out the invention. It should be understood that the illustrated embodiments are exemplary only, and should not be taken as limiting the scope of the invention.

We claim:

1. A baroreflex stimulation device comprising:
an electrode;
a controller including a signal generator coupled to the electrode, the controlling being operable to generate a sub-baroreflex threshold stimulus signal, the sub-baroreflex threshold stimulus signal being coupled to the electrode.

2. The device of claim 1, the electrode being incorporated into a collar configured to dispose the electrode adjacent the carotid sinus baroreceptor when the collar is disposed upon a patient's neck.

3. The device of claim 2, the collar being a portion of a garment.

4. The device of claim 3, the controller being disposed within the garment.

5. The device of claim 1, electrode comprising a thin-film sub-cutaneous electrode.

6. The device of claim 1, the electrode and controller configured for sub-cutaneous deployment.

6. The device of claim 1, the sub-baroreflex threshold stimulus signal being wirelessly coupled to the electrode.

7. The device of claim 6, electrode being externally deployed or sub-cutaneously deployed.

8. The device of claim 1, the electrode and controller being integral.

9. The device of claim 8, the device being externally deployed or sub-cutaneously deployed.

10. The device of claim 1, the stimulus comprising random noise components.

11. The device of claim 1, the stimulus configured to additively combine with a baroreflex generator potential.

12. The device of claim 1, the stimulus being of insufficient energy to directly stimulate baroreflex response.

13. The device of claim 1, the stimulus configured to create a stochastic resonance in a baroreflex generator potential.

14. A method of generating a baroreflex response comprising:
providing a sub-baroreflex response threshold stimulus signal; and
communicating the sub-baroreflex response threshold stimulus signal to a baroreceptor.

15. The method of claim 13, wherein communicating the sub-baroreflex response threshold stimulus signal to a baroreceptor comprises communicating the sub-baroreflex response threshold stimulus signal to a carotid sinus baroreceptor.

16. The method of claim 13, wherein communicating the sub-baroreflex response threshold stimulus signal to a baroreceptor comprises communicating the sub-baroreflex response threshold stimulus signal to a baroreceptor via an electrode.

17. The method of claim 13, wherein communicating the sub-baroreflex response threshold stimulus signal to a baroreceptor comprises communicating the sub-baroreflex response threshold stimulus signal to a baroreceptor via an electrode disposed sub-cutaneously.

18. The method of claim 13, wherein communicating the sub-baroreflex response threshold stimulus signal to a baroreceptor comprises communicating the sub-baroreflex response threshold stimulus signal to a baroreceptor via an electrode disposed externally.

19. The method of claim 13, wherein communicating the sub-baroreflex response threshold stimulus signal to a baroreceptor comprises communicating the sub-baroreflex response threshold stimulus signal to a baroreceptor via an electrode disposed within an article of clothing.

20. The method of claim 13, wherein communicating the sub-baroreflex response threshold stimulus signal to a baroreceptor comprises communicating the sub-baroreflex response threshold stimulus signal to a baroreceptor via an electrode disposed within a collar.

21. The method of claim 13, wherein providing a sub-baroreflex response threshold stimulus signal comprises providing a sub-baroreflex response threshold stimulus signal having random noise components.

22. The method of claim 13, wherein providing a sub-baroreflex response threshold stimulus signal comprises providing a sub-baroreflex response threshold stimulus signal comprising low amplitude noise.

23. The method of claim 13, wherein providing a sub-baroreflex response threshold stimulus signal comprises providing a sub-baroreflex response threshold stimulus signal that additively couples with a baroreceptor generator potential.

24. The method of claim 13, wherein providing a sub-baroreflex response threshold stimulus signal comprises providing a sub-baroreflex response threshold stimulus signal of insufficient energy to directly stimulate a baroreceptor response.

25. The method of claim 13, wherein providing a sub-baroreflex response threshold stimulus signal comprises providing a sub-baroreflex response threshold stimulus signal of insufficient energy to generate a stochastic resonance in a baroreflex generator potential.
